(19) United States
(12) Patent Application Publication
(10) Pub. No.: US 2021/0405032 A1
(43) Pub. Date: Dec. 30, 2021

(54) FUNCTIONAL VALIDATION OF DONOR IMMUNE-REGULATORY CELLS

(71) Applicant: CROWN SCIENTIFIC, LLC, San Antonio, TX (US)

(72) Inventor: Ramon Coronado, San Antonio, TX (US)

(21) Appl. No.: 17/360,886

(22) Filed: Jun. 28, 2021

Related U.S. Application Data
(60) Provisional application No. 63/045,598, filed on Jun. 29, 2020.

(51) Int. Cl. G01N 33/50

(52) U.S. Cl. CPC G01N 33/5091

(57) ABSTRACT

The invention provides methods for the validation of donor immune-regulatory cells. The validation method includes a combination of assays to determine the capability of immune-regulatory cells to immune-regulate adaptive and innate immune system components.

This application claims benefit of priority under 35 U.S.C. § 119(e) of U.S. Ser. No. 63/045,598, filed Jun. 29, 2020, the entire contents of which is incorporated herein by reference in its entirety.

### BACKGROUND OF THE INVENTION

### Field of the Invention

The present invention relates generally to the functional validation of donor immune-regulatory cells, and more specifically to donor immune-regulatory cell lot release criterion for therapeutic use.

### Background Information

In the current mesenchymal stromal cells (MSCs) manufacturing industry there is a component lacking for the validation of donor MSCs, which is necessary for regulatory compliance under FDA requirements. Lot release criterion is required by the FDA for a drug product or biologic to allow manufacturers to corroborate, validate and demonstrate that the drug product or biologic shows an acceptable therapeutic functional effect and to monitor the quality of donor the drug product or biologic. Lot release criterion for donor MSCs is required to monitor the quality of donor MSCs, especially as collection and/or manufacturing protocols might change over time and due to donor variability. As is required for manufacturing a therapeutic enzyme, “units” of activity need to be determined before MSCs are available for therapeutic use to demonstrate that the cells are MSCs and that the MSCs have acceptable activity and functionality.

In the MSC field, many companies are now developing “lot release criterion” based on multiple factors, including: MSC protein markers, gene characteristics and differentiation potential. However, these criteria do not validate the functionality of MSCs, specifically in the context in which such cells provide their therapeutic effect, i.e. immune-regulation of cells of the adaptive and innate immune system.

### SUMMARY OF THE INVENTION

The present invention is based on the seminal discovery that a combination of assays which determine the capability of immune-regulatory cells such as MSCs to immune-regulate both adaptive and innate immune system components can be used as a donor MSC lot release criterion for the functional validation of donor MSCs for therapeutic use, and as a potency assay to validate therapeutic effect.

In one embodiment, the present invention provides a method of validating donor immune-regulatory cell activity including determining the ability of immune-regulatory cells to immune-regulate adaptive immune cells and innate immune cells, wherein the immune-regulatory cells are validated if the immune-regulatory cells immune-regulate both adaptive immune system cells and innate immune system cells. In various aspects, the immune-regulatory cells are mesenchymal stromal cells (MSC). In many aspects, the validation of donor MSC activity is a lot release criterion for MSC therapeutic use.

In one aspect, the invention includes determining the ability of MSCs to inhibit lymphocyte proliferation. In various aspects, the inhibition of lymphocyte proliferation includes co-culturing MSCs and CD3+ lymphocytes, activating the CD3+ lymphocytes with a T cell receptor (TCR) activator, and determining an EC50 response. In some aspect, the MSCs and CD3+ lymphocytes are co-cultured at a 1:1 ratio. In other aspects, the TCR activator is selected from the group consisting of CD3, CD28, CD2 or a combination thereof. In one aspect, the EC50 response indicates inhibition of lymphocyte proliferation.

In another aspect, the invention includes determining the ability of MSCs to inhibit proliferation and maturation of B cells. In yet another aspect, the invention includes determining the ability of MSCs to inhibit B cell proliferation and antibody production. In various aspects the inhibition of B cell maturation and proliferation and/or the inhibition of antibody production includes incubating MSCs with B cells, activating the B cells with antigens, and determining the inhibition of B cell maturation, proliferation, and antibody production. In various aspects, the inhibition of B cell maturation and/or the inhibition of antibody production includes determining the expression level of CD19, CD20, CD27, CD38, CD138, IgG, IgM, IgE, IgA. In certain aspects a decreased expression of CD19, CD20, CD27, CD138, CD38, IgG, IgM, IgE, IgA indicates an inhibition of B cell maturation and/or an inhibition of antibody production, in various aspects, the expression of CD19 is inhibited at least 50% as compared to a control. In one aspect, determining the inhibition of B cell maturation and proliferation is by flow cytometry.

In another aspect, the invention provides a method of determining the ability of MSCs to inhibit monocyte maturation and proliferation. In various aspects, the inhibition of monocyte maturation and proliferation includes incubating MSCs with monocytes, activating the monocytes with a toxin, and determining the inhibition of monocyte maturation and proliferation. In one aspect, the inhibition of monocyte maturation includes determining the expression level of CD1a, CD11b, CD11c, CD14, CD40, CD80, CD83, CD86 and/or MEW II. In various aspects, a decreased expression of CD1a, CD11b, CD11c, CD14, CD40, CD80, CD83, CD86 and/or MEW II indicates an inhibition of monocytes maturation. In other aspects, the expression of CD1a is inhibited at least 50% as compared to a control. In another aspect, determining the EC50 inhibition of monocyte maturation and proliferation is by flow cytometry.

In an additional aspect, the method of functionally validating mesenchymal stromal cell (MSC) activity further includes determining the expression level of molecular markers. In various aspects, the molecular markers are selected from the group including CD105, CD73, CD90, CD45, CD34, CD14, CD11b, CD79 alpha, CD19, HLA-DR and a combination thereof.

In another embodiment, the present invention provides for an immune-regulatory cell lot release criterion for determining that donor immune regulatory cells immune-regulate both adaptive immune system cells and innate immune system cells. In various aspect, the immune regulator cells are MSCs. In one aspect, immune regulation of adaptive immune system cells includes determining the ability of the MSCs to inhibit lymphocyte proliferation. In another aspect, immune regulation of adaptive immune system cells includes determining the ability of the MSCs to inhibit proliferation and maturation of B cells. In yet another aspect, immune regulation of adaptive immune system cells includes determining the ability of the MSCs to inhibit B cell proliferation and antibody production. In one aspect, the ability of MSCs to immune regulate innate immune system cells comprises determining the ability of the MSCs to inhibit monocyte maturation and proliferation.

In one embodiment, the present invention provides for a method of determining an immunological effect of mesenchymal stromal cells (MSCs) to immune regulate innate immune system cells and adaptive immune system cells. In one aspect, the determining the ability of MSCs to immune-regulate adaptive immune system cells is performed by determining the ability of MSCs to inhibit lymphocyte proliferation. In certain aspects, the inhibition of lymphocyte proliferation is determined by co-culturing MSCs with CD3+ lymphocytes; activating the CD3+ lymphocytes with a T cell receptor (TCR) activator; and determining an EC50 response. In another aspect, the determining the ability of MSCs to immune-regulate adaptive immune system cells is performed by determining the ability of MSCs to inhibit proliferation and maturation of B cells. In yet another aspect, the determining the ability of MSCs to immune-regulate adaptive immune system cells is performed by determining the ability of MSCs to inhibit B cell proliferation and antibody production. In various aspect, the inhibition of B cell maturation and proliferation and/or the inhibition of antibody production is determined by incubating MSCs with B cells, activating the B cells with antigens, and determining the inhibition of B cell maturation, proliferation, and antibody production. In an additional aspect, the determining the ability of MSCs to immune regulate innate immune system cells involves determining the ability of MSCs to inhibit monocyte maturation and proliferation. In a further aspect, the inhibition of monocyte maturation and proliferation is determined by incubating MSCs with monocytes; activating the monocytes with a toxin; and determining the inhibition of monocyte maturation and proliferation.

### BRIEF DESCRIPTION OF THE DRAWINGS

FIG. 1 is a graphical representation of an MSC lot validation assay using adaptive immune system cells.

FIG. 2 is a graphical representation of an MSC lot validation assay using innate immune system cells.

### DETAILED DESCRIPTION OF THE INVENTION

The present invention is based on the seminal discovery that a combination of assays which determines the capability of immune-regulatory cells such as MSCs to immune-regulate both adaptive and innate immune system components can be used as a donor MSC lot release criterion for the functional validation of donor MSC for therapeutic use, and as a potency assay to validate therapeutic effect.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a method of validating donor immune-regulatory cell activity including determining the ability of immune-regulatory cells to immune-regulate adaptive immune cells and innate immune cells, wherein the immune-regulatory cells are validated if the immune-regulatory cells immune-regulate both adaptive immune system cells and innate immune system cells.

Immune regulation is fundamental to ensure that an immune response is appropriate. As used herein, “immune-regulatory cells” refers to cell populations that are responsible for maintaining a balanced and appropriate immune response. Immune-regulatory cells include regulatory T cells, B cells and macrophages, as well as myeloid-derived suppressor cells, dendritic cells and mesenchymal stromal cells (MSCs). These cells can modulate immune responses by inhibiting effector cells and by inducing other regulatory cells.

In various aspects, the immune-regulatory cells are mesenchymal stromal cells. Mesenchymal stromal cells (MSCs) are spindle-shaped plastic adherent cells that can be isolated from many tissues including bone marrow and adipose. MSCs have multipotent differentiation capacity in vitro and can be treated to generate differentiated cells such as osteoblasts, adipocytes and chondrocytes in vitro, but do not have the capacity to reconstitute an entire organ. In the last decade, MSCs have been extensively studied for their potential role in regenerative medicine, immune-regulation, neuroprotection, and anti-tumor effect.

In another aspect, the validation of donor MSC activity is an important criterion for MSC therapeutic use.

The term “MSC therapeutic use” is used interchangeably herein with the term “MSC treatment” and refers to both 1) therapeutic treatments or measures using MSCs to cure, slow down, lessen symptoms of, and/or halt progression of a diagnosed pathologic conditions or disorder that can be treated by MSCs, and 2) and prophylactic/preventative measures using MSCs. Those in need of treatment may include individuals already having a particular medical disorder as well as those who may ultimately acquire the disorder (i.e., those needing preventive measures).

As used herein, “MSCs”, “MSC lot”, “MSC batch” and the like refer to the MSCs isolated from a donor. As used herein, a MSC lot can be “tested”, “evaluated” or “validated”, to assess the properties of the MSCs.

MSCs are extensively studied for their potential role in regenerative medicine, immune-regulation, neuroprotection, and anti-tumor effect. However, the clinical use of MSCs as therapeutic agent requires the isolation of pure MSCs as well as standardized methods for their definition and characterization. Such methods are essential for the validation of MSC lot and for the release of said lot for therapeutic use.

In the MSC field, the current validation criteria used as are based on multiple factors, mainly using: MSC protein markers, gene characteristics and differentiation potential. Each lot is tested to evaluate if the cells retain MSC characteristics and must meet those criteria to be validated.

The International Society for Cellular Therapy has developed minimal experimental criteria that constitute MSC validation standards. Those criteria rely upon a phenotypic verification of MSCs, along with a functional verification of MSCs. Phenotypic verification of MSCs includes adherence to plastic in standard culture conditions and the evaluation of positive MSC markers that must be expressed by more than 95% of the MSC population, and of negative markers that must be expressed by less than 2% of the MSC population as measured, for example, by flow cytometry. Positive markers include CD73/5′-Nucleotidase, CD90/Thy1 and CD105/Endoglin. Negative markers include CD34, CD45, CD11b/Integrin alpha M or CD14, CD79 alpha or CD19 and HLA-DR. Functional verification of MSCs relies on the assessment of the ability of the cells to differentiate into adipocytes, chondrocytes, and osteocytes in culture using a combination of media and supplements. However, new knowledge supports the hypothesis that such cells do not provide their therapeutic effect by grafting and differentiating into other cell types hence the markers and differentiation capability has little to no significance when developing functional assays that reflect their therapeutic potency.

However, since there is no MSC-specific marker, methods such as positive and negative selection limits the purity of isolated MSCs, which generates populations of cells that differ in their growth kinetics and differentiation potential. Moreover, none of these assays can functionality validate MSCs for therapeutic use, and thus, these assays alone cannot be considered as a lot release criterion for therapeutic use of MSCs with potent therapeutic effect.

The functional validation method of MSC lot of the present invention is a combination of assays that determine the capability of the MSCs to immune-regulate adaptive immune system components and innate immune system components. The ability of MSCs to have these functions is essential to demonstrate that the MSC lot is acceptable for therapeutic use.

In one aspect, determining the ability of MSCs to immune-regulate adaptive immune cells includes determining the ability of MSCs to inhibit lymphocyte proliferation. In another aspect, the invention includes determining the ability of MSCs to inhibit proliferation and maturation of B cells. In yet another aspect, the invention includes determining the ability of MSCs to inhibit B cell proliferation and antibody production. In another aspect, determining the ability of MSCs to immune-regulate innate immune cells includes determining the ability of MSCs to inhibit monocyte maturation and proliferation.

In one aspect, determining the ability of MSCs to immune-regulate adaptive immune cells includes determining the ability of MSCs to induce T regulatory cells. In one aspect, determining the ability of MSCs to immune-regulate adaptive immune cells includes determining the ability of MSCs reduce Th17 proliferation. In one aspect, determining the ability of MSCs to immune-regulate adaptive immune cells includes determining the ability of MSCs modulate the Th1/Th2 balance. In another aspect, determining the ability of MSCs to immune-regulate innate immune cells includes determining the ability of MSCs to induce tolerogenic dendritic cells. In another aspect, determining the ability of MSCs to immune-regulate innate immune cells includes determining the ability of MSCs to induce Macrophage type 2. In another aspect, determining the ability of MSCs to immune-regulate innate immune cells includes determining the ability of MSCs to reduce B cell antigen production. In another aspect, determining the ability of MSCs to immune-regulate innate immune cells includes determining the ability of MSCs to induce B regulatory cells. In another aspect, determining the ability of MSCs to immune-regulate innate immune cells includes determining the ability of MSCs to regulate NK cells.

In one aspect, determining the ability of MSCs to immune-regulate immune cells includes determining the ability of MSCs to regulate viral load uptake.

The immune system is a system of biological structures and processes within an organism that protects against disease. This system is a diffuse, complex network of interacting cells, cell products, and cell-forming tissues that protects the body from pathogens and other foreign substances, destroys infected and malignant cells, and removes cellular debris: the system includes the thymus, spleen, lymph nodes and lymph tissue, stem cells, white blood cells, antibodies, and lymphokines. The immune system is classified into two subsystems, the innate immune system and the adaptive immune system.

The innate immune system is the non-specific immune system that includes cells and mechanisms involved in the defense of the host from infection. In vertebrate, it includes anatomical barrier such as the skin, the complement system, and white blood cells such as natural killer cells, mast cells, eosinophils, basophils, macrophages, neutrophils, and dendritic cells.

The adaptive immune system or acquired immune system is the antigen-specific immune system that requires the recognition of non-self antigens to generate a response. In vertebrate, it includes highly specialized, systemic cells and processes that eliminate pathogens. Adaptive immune cells comprise B and T lymphocytes. Naive B and T lymphocytes are cells that have not matured yet, effector cells are cells that have been activated by their cognate antigen, and are actively involved in eliminating a pathogen, memory cells are the survivors of past infections that remains to induce faster response to same antigens.

In various aspects, the inhibition of lymphocyte proliferation includes co-culturing MSCs and CD3+ lymphocytes, activating the CD3+ lymphocytes with a T cell receptor (TCR) activator, and determining an EC50 response. In some aspect, the MSCs and CD3+ lymphocytes are co-cultured at a 1:1 ratio. In other aspects, the TCR activator is selected from the group consisting of CD3, CD28, CD2 or a combination thereof. In one aspect, the EC50 response indicates inhibition of lymphocyte proliferation.

The T-cell receptor, or TCR, is a molecule found on the surface of T cells, or T lymphocytes and that is responsible for recognizing fragments of antigen such as peptides bound to major histocompatibility complex (MHC) molecules. When the TCR engages with antigenic peptide and MHC (peptide/MHC), the T lymphocyte is activated through signal transduction, which leads to T cells proliferation and to cytokines production. CD3/CD28/CD2 T cell activator is a commercial CD3+ T cell activator designed to activate and expand human T cells in the absence of magnetic beads, feeder cells or antigen.

The ability of MSCs to inhibit immune cell proliferation is measured by the determination of an EC50. The “EC50” is the half maximal effective concentration (EC50), as used herein, an EC50 refers to the concentration of cells which is sufficient to induce inhibition of proliferation halfway between the baseline and maximum after a specified exposure time. The EC50 value of the present invention specifically refers to the concentration of immune-regulatory cells which is sufficient to induce the inhibition of T cell proliferation, CD1a expression, or antibody production for example. The EC50 corresponding to an immune-regulatory cell lot, as determined during a clinical trial, reflects the number of immune-regulatory cells from this lot that are required to show statistical effectiveness. This criterion is then used during the commercialization process to validate an immune-regulatory cell lot prior to its release: a lot that only presents statistical effectiveness with more cells than determined by the EC50 is not validated, however if a lot presents a statistical effectiveness with an equivalent or smaller amount of cells is validated and can be released. As used herein, the EC50 can be analyzed for T cell proliferation, CD1a expression, B cell antigen production etc.

In various aspects the inhibition of B cell maturation and proliferation and/or the inhibition of antibody production includes incubating MSCs with B cells, activating the B cells with antigens, and determining the inhibition of B cell maturation, proliferation, and antibody production. In various aspects, the inhibition of B cell maturation and/or the inhibition of antibody production includes determining the expression level of CD19, CD20, CD27, CD38, CD138, IgG, IgM, IgE, IgA. In certain aspects a decreased expression of CD19, CD20, CD27, CD138, CD38, IgG, IgM, IgE, IgA indicates an inhibition of B cell maturation and/or an inhibition of antibody production, in various aspects, the expression of CD19 is inhibited at least 50% as compared to a control. In one aspect, determining the inhibition of B cell maturation and proliferation is by flow cytometry.

In various aspects, the inhibition of monocyte maturation, tolerogenic dendritic cells induction and proliferation includes incubating MSCs with monocytes, activating the monocytes with a toxin, and determining the inhibition of monocyte maturation, tolerogenic induction and proliferation. In one aspect, the inhibition of monocyte maturation or tolerogenic dendritic cell induction includes determining the expression level of CD1a, CD11b, CD11c, CD14, CD40, CD80, CD83, CD86 and/or MHC II. In various aspects, a decreased expression of CD1a, CD11b, CD11c, CD14, CD40, CD80, CD83, CD86 and/or MHC II indicates an inhibition of monocytes maturation. In other aspects, the expression of CD1a is inhibited at least 50% as compared to a control. In another aspect, determining the inhibition of monocyte maturation and proliferation is by flow cytometry.

In an additional aspect, the method of validating mesenchymal stromal cell (MSC) activity further includes determining the expression level of molecular markers. In various aspects, the molecular markers are selected from the group including CD105, CD73, CD90, CD45, CD34, CD14, CD11b, CD79 alpha, CD19, HLA-DR and a combination thereof.

In another embodiment, the present invention provides for an immune-regulatory cell lot release criterion for determining that donor immune regulatory cells immune-regulate both adaptive immune system cells and innate immune system cells. In various aspect, the immune regulator cells are MSCs. In one aspect, immune regulation of adaptive immune system cells includes determining the ability of the MSCs to inhibit lymphocyte proliferation. In another aspect, immune regulation of adaptive immune system cells includes determining the ability of the MSCs to inhibit proliferation and maturation of B cells. In yet another aspect, immune regulation of adaptive immune system cells includes determining the ability of the MSCs to inhibit B cell proliferation and antibody production. In one aspect, the ability of MSCs to immune regulate innate immune system cells comprises determining the ability of the MSCs to inhibit monocyte maturation and proliferation.

In one embodiment, the present invention provides for a method of determining an immunological effect of mesenchymal stromal cells (MSCs) to immune regulate innate immune system cells and adaptive immune system cells. In one aspect, the determining the ability of MSCs to immune-regulate adaptive immune system cells is performed by determining the ability of MSCs to inhibit lymphocyte proliferation. In certain aspects, the inhibition of lymphocyte proliferation is determined by co-culturing MSCs with CD3+ lymphocytes; activating the CD3+ lymphocytes with a T cell receptor (TCR) activator; and determining an EC50 response. In another aspect, the determining the ability of MSCs to immune-regulate adaptive immune system cells is performed by determining the ability of MSCs to inhibit proliferation and maturation of B cells. In yet another aspect, the determining the ability of MSCs to immune-regulate adaptive immune system cells is performed by determining the ability of MSCs to inhibit B cell proliferation and antibody production. In various aspect, the inhibition of B cell maturation and proliferation and/or the inhibition of antibody production is determined by incubating MSCs with B cells, activating the B cells with antigens, and determining the inhibition of B cell maturation, proliferation, and antibody production. In an additional aspect, the determining the ability of MSCs to immune regulate innate immune system cells involves determining the ability of MSCs to inhibit monocyte maturation and proliferation. In a further aspect, the inhibition of monocyte maturation and proliferation is determined by incubating MSCs with monocytes; activating the monocytes with a toxin; and determining the inhibition of monocyte maturation and proliferation.

Presented below are examples discussing methods to validate a MSC lot that retains immune-regulative functional properties, contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used

### EXAMPLES

### Example 1

### Lymphocyte Proliferation after Co-Culture with Mesenchymal Stromal Cells

In order to validate that a donor batch of MSCs has adequate immune-regulative therapeutic activity, the ability of MSCs to inhibit the proliferation of adaptive immune system cells is evaluated.

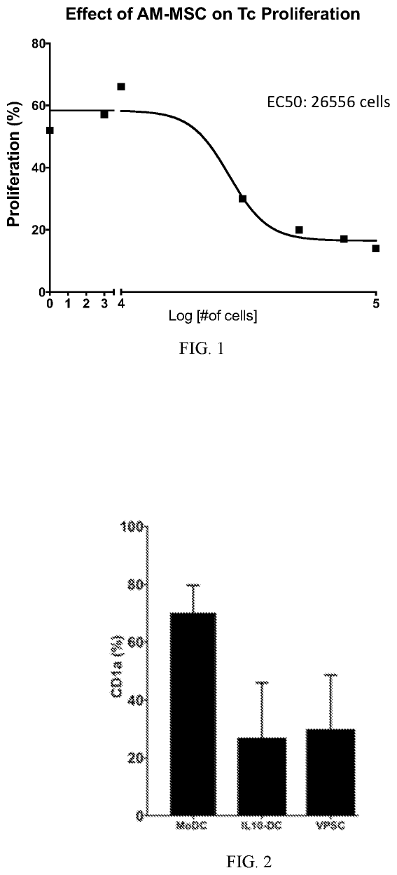

MSCs are co-cultured with CD3+ lymphocytes in various ratios. The lymphocytes are then activated by incubation with a TCR activator (for example, CD3, CD28, CD2). The half maximal response (EC50) is determined by plotting the number of cells that proliferate versus the number of cells that do not proliferate. A donor MSC lot is validated by the ability of the MSCs to inhibit lymphocyte proliferation at a maximum of 1:1 MSC:lymphocyte ratio. The EC50 curve shown in FIG. 1 is a characteristic curve for a donor MSC batch to donor MSC batch variability test. The EC50 value refers to the concentration of cells which is sufficient to induce inhibition of a desired effect, here T cell proliferation. In this example, the amniotic mesenchymal stromal cells (AM-MSCs) presented an EC50 of 25,000 cells±5,000 cells during the clinical trial, and showed statistical effectiveness, these criteria can be used for validating AM-MSC lot release during commercialization. Using this criterion, an AM-MSC lot presenting statistical effectiveness at inhibiting T cell proliferation with 25,000 cells±5,000 cells or less will be validated and released, while an AM-MSC lot presenting a statistical effectiveness at inhibiting T cell proliferation only with more than 25,000 cells±5,000 cells won't be released for therapeutic use.

By showing the effects of MSCs on immune cells from the adaptive immune system, the ability of the cells to immune-regulate is determined and a donor MSC lot can be functionally validated.

### Example 2

### Monocyte Proliferation and Maturation after Co-Culture with Mesenchymal Stromal Cells

In order to validate that a donor batch of MSCs has adequate immune-regulative therapeutic activity, the ability of MSCs to inhibit the proliferation and maturation of innate immune system cells is evaluated.

MSCs are co-cultured with monocytes and the monocytes are activated or differentiated to dendritic cells using GM-CSF, IL4 and a final activation with a toxin such as Lipopolysaccharide, or cytokines (IL6, IFNg, TNFa etc.). The proliferation and maturation of the monocytes into mature dendritic cells is then evaluated by flow cytometry by measuring the expression levels of CD1a and CD83, two cell surface markers expressed by monocyte-derived mature dendritic cells. As illustrated in FIG. 2, a MSC lot that inhibits the expression of CD1a at least by 50% compared to control monocyte-derived mature dendritic cells (MoDC) and induces a tolerogenic dendritic cell profile evident by the upregulation of CD85d.

By showing the effects of MSCs on immune cells from both the innate and the adaptive (see Example 1 above) immune systems, a MSC lot demonstrates its capability to immune-regulate and is thus functionally validated. Such MSC-functionally-validated lot meets the lot release criterion and can be used as a therapeutic agent.

Although the invention has been described with reference to the above examples, it will be understood that modifications and variations are encompassed within the spirit and scope of the invention. Accordingly, the invention is limited only by the following claims.

What is claimed is:

1. A method of validating immune-regulatory cell activity comprising determining the ability of immune-regulatory cells to immune-regulate adaptive immune system cells and innate immune system cells, wherein the immune-regulatory cells are validated if the immune-regulatory cells immune regulate both adaptive immune system cells and innate immune system cells.

2. The method of claim 1, wherein the immune-regulatory cells are mesenchymal stromal cells (MSC).

3. The method of claim 2, wherein determining the ability of MSCs to immune-regulate adaptive immune system cells comprises determining the ability of MSCs to inhibit lymphocyte proliferation.

4. The method of claim 3, wherein the inhibition of lymphocyte proliferation comprises:
a) co-culturing MSCs with CD3+ lymphocytes;
b) activating the CD3+ lymphocytes with a T cell receptor (TCR) activator; and
c) determining an EC50 response.

5. The method of claim 4, wherein the MSCs and CD3+ lymphocytes are co-cultured at a 1:1 ratio; wherein the TCR activator is selected from the group consisting of CD3, CD28, CD2 or a combination thereof and wherein the EC50 response indicates inhibition of lymphocyte proliferation.

6. The method of claim 2, wherein determining the ability of MSCs to immune regulate adaptive immune system cells comprises determining the ability of MSCs to inhibit proliferation and maturation of B cells and B cell antibody production and wherein determining the ability of MSCs to immune regulate innate immune system cells comprises determining the ability of MSCs to inhibit monocyte maturation and proliferation and/or to induce tolerogenic dendritic cells.

7. The method of claim 6, wherein the inhibition of B cell maturation and proliferation and/or the inhibition of antibody production comprises:
a) incubating MSCs with B cells;
b) activating the B cells with antigens; and
c) determining the inhibition of B cell maturation, proliferation, and antibody production.

8. The method of claim 7, wherein a decreased expression of CD19, CD20, CD27, CD138, CD38, IgG, IgM, IgE, IgA indicates an inhibition of B cell maturation and/or an inhibition of antibody production.

9. The method of claim 6, wherein the inhibition of monocyte maturation and proliferation comprises:
a) incubating MSCs with monocytes;
b) activating the monocytes with a toxin; and
c) determining the inhibition of monocyte maturation and proliferation.

10. The method of claim 9, wherein a decreased expression of CD1a, CD11b, CD11 c, CD14, CD40, CD80, CD83, CD86 and/or MEW II indicates an inhibition of monocytes maturation.

11. The method of claim 2, further comprising determining the expression level of molecular markers selected from the group comprising CD105, CD73, CD90, CD45, CD34, CD14, CD11b, CD79alpha, CD19, HLA-DR and a combination thereof.

12. An immune-regulatory cell lot release criterion comprising determining that donor immune regulatory cells immune-regulate both adaptive immune system cells and innate immune system cells.

13. The lot release criterion of claim 12, wherein the immune regulator cells are MSCs.

14. The lot release criterion of claim 13, wherein immune regulation of adaptive immune system cells comprises determining the ability of the MSCs to inhibit lymphocyte proliferation, to inhibit proliferation, and maturation of B cells and/or to inhibit B cell antibody production and wherein the ability of MSCs to immune regulate innate immune system cells comprises determining the ability of the MSCs to inhibit monocyte maturation and proliferation.

15. A method of determining an immunological effect of mesenchymal stromal cells (MSCs) to immune regulate innate immune system cells and adaptive immune system cells.

16. The method of claim 15, wherein determining the ability of MSCs to immune-regulate adaptive immune system cells comprises determining the ability of MSCs to inhibit lymphocyte proliferation.

17. The method of claim 16, wherein the inhibition of lymphocyte proliferation comprises:
a) co-culturing MSCs with CD3+ lymphocytes;
b) activating the CD3+ lymphocytes with a T cell receptor (TCR) activator; and
c) determining an EC50 response.

18. The method of claim 15, wherein determining the ability of MSCs to immune regulate adaptive immune system cells comprises determining the ability of the MSCs to inhibit proliferation and maturation of B cells.

19. The method of claim 18, wherein the inhibition of B cell maturation and proliferation and/or the inhibition of antibody production comprises:
a) incubating MSCs with B cells;
b) activating the B cells with antigens; and
c) determining the inhibition of B cell maturation, proliferation, and antibody production.

20. The method of claim 15, wherein determining the ability of MSCs to immune regulate innate immune system cells comprises determining the ability of MSCs to inhibit monocyte maturation and proliferation.
